(19) United States
(12) Patent Application Publication
(10) Pub. No.: US 2026/0183484 A1
(43) Pub. Date: Jul. 2, 2026

(54) A REUSABLE DRIVE UNIT OF A MEDICAMENT DELIVERY DEVICE

(71) Applicant: SHL Medical AG, Zug (CH)

(72) Inventors: Daniel Carlsson, Nacka Strand (SE); Daniel Säll, Nacka Strand (SE)

(21) Appl. No.: 19/125,880

(22) PCT Filed: Oct. 20, 2023
(86) PCT No.: PCT/EP2023/079218

§ 371 Date: Apr. 30, 2025

(30) Foreign Application Priority Data
Nov. 4, 2022 (EP) 22205507.1

(51) Int. Cl. A61M 5/20; A61M 5/315

(52) U.S. Cl. CPC A61M 5/2033; A61M 5/31578; A61M 2005/2013; A61M 2005/206; A61M 2005/208; A61M 2005/31588; A61M 2202/0007; A61M 2205/103; A61M 2205/12

(57) ABSTRACT

The present disclosure provides a reusable drive unit of a medicament delivery device. The medicament delivery device comprises the reusable drive unit and a disposable cassette releasably attached to the reusable drive unit. The reusable drive unit includes a housing configured to accommodate a portion of the disposable cassette. The housing comprises an inner wall. The reusable drive unit further includes a lead screw extending along a longitudinal axis between a proximal end and a distal end within the housing. The lead screw comprises a thread extending between the proximal end and the distal end. The reusable drive unit further includes a distal nut attached to the distal end of the lead screw. The distal nut comprises a surface facing in a direction transverse to the longitudinal axis. The surface is adjacent to the inner wall of the housing. The distal nut comprises a non-circular cross-section perpendicular to the longitudinal axis and the inner wall of the housing comprises a non-circular cross-section perpendicular to the longitudinal axis such that the distal nut and the lead screw are rotationally fixed to the housing. The reusable drive unit further includes a driver rotatable around the lead screw within the housing. The rotation of the driver around the lead screw is configured to move the lead screw proximally so that a stopper of a medicament container in the disposable cassette is moved by the lead screw.

### TECHNICAL FIELD

The present disclosure generally relates to a reusable drive unit of a medicament delivery device, and particularly to a reusable drive unit of a medicament delivery device, the reusable drive unit comprising a lead screw and a driver configured to rotate around the lead screw.

### BACKGROUND

Medicament delivery devices such as pen-type manual injectors or auto-injectors are generally known for self-administration of a medicament by patients without formal medical training. For example, patients suffering from diabetes may require repeated injections of insulin, or patients may require regular injections of other types of medicaments, such as growth hormones.

It is an advantage when the medicament delivery devices for self-administration comprise automatic functions so that even if users are without professional training or are in an emergency situation, the users can easily and properly use the medicament delivery devices.

Even though many of the devices on the market, as well as the ones described above, have their respective advantages, there is still room for improvement.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a reusable drive unit of a medicament delivery device, wherein the medicament delivery device comprises the reusable drive unit and a disposable cassette releasably attached to the reusable drive unit, the reusable drive unit comprising: a housing configured to accommodate a portion of the disposable cassette. The housing comprises an inner wall. The reusable drive unit comprises a lead screw extending along a longitudinal axis between a proximal end and a distal end within the housing. The lead screw comprises a thread extending between the proximal end and the distal end. A distal nut is attached to the distal end of the lead screw. The distal nut comprises a surface facing in a direction transverse to the longitudinal axis. The surface is adjacent to the inner wall of the housing. The distal nut comprises an outer surface having a non-circular cross-section perpendicular to the longitudinal axis and the inner wall of the housing comprises an inner surface having a non-circular cross-section perpendicular to the longitudinal axis such that the distal nut and the lead screw are rotationally fixed to the housing.

A driver is rotatable around the lead screw within the housing. The driver is engaged with the lead screw via a threaded engagement. A first switch is arranged within the housing. Upon activation of the first switch, the rotation of the driver around the lead screw is configured to move the lead screw proximally so that a stopper of a medicament container in the disposable cassette is moved by the lead screw.

Preferably, according to another embodiment, the driver is a motor electrically connected to the first switch.

Preferably, according to another embodiment, the motor comprises a rotor and a channel extending through the rotor. The lead screw is partially positioned within the channel.

Preferably, according to another embodiment, the channel comprises a thread extending around the channel. The thread is engaged with the thread of the lead screw.

Alternatively, according to another embodiment, the reusable drive unit comprises a connector operably connected to the rotor such that the connector is configured to be rotated by the rotor.

Preferably, according to another embodiment, a channel extends through the connector and is aligned with the channel of the rotor along the longitudinal axis.

Preferably, according to another embodiment, the channel of the connector comprises a thread extending around the channel of the connector. The thread is engaged with the thread of the lead screw.

Preferably, according to another embodiment, the connector is attached to the rotor.

Preferably, according to another embodiment, the connector and the rotor are made of different materials.

Preferably, according to another embodiment, the connector is made of plastic.

Preferably, according to another embodiment, the rotor is made of metal.

Preferably, according to another embodiment, the longitudinal axis is the central axis of the motor.

Preferably, according to another embodiment, an engagement formed between the distal nut and the inner wall of the housing is a ribs-ribs engagement or a ribs-grooves engagement.

Preferably, according to another embodiment, the distal nut comprises a polygonal cross-section perpendicular to the longitudinal axis and the inner wall of the housing comprises a polygonal cross-section perpendicular to the longitudinal axis.

Preferably, according to another embodiment, the driver is configured to reciprocate the movement of the lead screw.

Preferably, according to another embodiment, the reusable drive unit comprises a second switch accommodated within the housing.

Preferably, according to another embodiment, the second switch is aligned with the distal nut in the direction of the longitudinal axis.

Preferably, according to another embodiment, the second switch is positioned distally away from the distal nut.

Preferably, according to another embodiment, the second switch is configured to be switched by the distal nut when the lead screw is moved distally relative to the driver such that when the second switch is switched the motor is stopped.

Preferably, according to another embodiment, the second switch is a non-contact switch.

Alternatively, according to another embodiment, the second switch is configured to be switched by being pushed in the distal direction relative to the housing by the distal nut.

Preferably, according to another embodiment, the second switch comprises a flipping arm.

Preferably, according to another embodiment, the second switch is configured to be switched when the flipping arm is pivoted by the distal nut.

Preferably, according to another embodiment, the housing extends along the longitudinal axis between a proximal end and a distal end.

Preferably, according to another embodiment, the housing comprises a fixture at a proximal portion of the housing.

Preferably, according to another embodiment, the fixture is configured to be releasably attached to a counter fixture of the disposable cassette of the medicament delivery device.

Preferably, according to another embodiment, the fixture and the counter fixture form a bayonet connection or a thread connection.

Preferably, according to another embodiment, the reusable drive unit comprises an activator movable relative to the housing in the direction of the longitudinal axis between a proximal position where the first switch is deactivated and a distal position where the first switch is activated by the activator.

Preferably, according to another embodiment, the first switch is a non-contact switch.

Alternatively, according to another embodiment, the activator comprises a first surface. The first surface of the activator is engaged with the first switch in the distal position and is disengaged with the first switch in the proximal position.

Preferably, according to another embodiment, the activator is configured to be moved from the proximal position to the distal position by a component of the disposable cassette of the medicament delivery device when the disposable cassette is attached to the housing.

Preferably, according to another embodiment, the activator comprises a proximally directed surface configured to be in contact with a distally directed surface of the component of the disposable cassette.

Preferably, according to another embodiment, the proximally directed surface of the activator is bevelled.

Preferably, according to another embodiment, the reusable drive unit comprises a third switch and a communication unit connected to the third switch.

Preferably, according to another embodiment, the communication unit is configured to receive information from the disposable cassette.

Preferably, according to another embodiment, the communication unit is configured to be activated when the third switch is activated. The third switch is configured to be operably activated by a component of the disposable cassette of the medicament delivery device when the disposable cassette is attached to the housing.

Preferably, according to another embodiment, the third switch is a non-contact switch.

Alternatively, according to another embodiment, the third switch is operably movable by a component of the disposable cassette of the medicament delivery device when the disposable cassette is attached to the housing between an active position where the third switch is activated and an inactive position where the third switch is deactivated.

Preferably, according to another embodiment, the activator comprises a second surface. The activator is movable in the direction of the longitudinal axis between a further proximal position where the second surface of the activator is spaced apart from the third switch and the proximal position where the second surface of the activator is engaged with the third switch such that the third switch is activated.

Preferably, according to another embodiment, the reusable drive unit is used in a medicament delivery device.

Preferably, according to another embodiment, the medicament container of the medicament delivery device is a syringe, a cartridge or a collapsible bag.

Preferably, according to another embodiment, the medicament container of the medicament delivery device is made of glass material or plastic material.

Preferably, according to another embodiment, the medicament container of the medicament delivery device is accommodated within the disposable cassette.

Preferably, according to another embodiment, the medicament delivery device is an injection device, an inhalation device, or a medical sprayer.

Preferably, according to another embodiment, the medicament delivery device is an auto-injector.

Preferably, according to another embodiment, the medicament delivery member is an injection needle or a spray nozzle.

Preferably, according to another embodiment, the medicament delivery member is accommodated in the disposable cassette.

Preferably, according to another embodiment, the medicament delivery device comprises a disposable cassette releasably attached to the reusable drive unit.

Preferably, according to another embodiment, the disposable cassette is configured to be fully received within the housing of the reusable drive unit.

Preferably, according to another embodiment, the disposable cassette is configured to be partially received within the housing of the reusable drive unit.

Preferably, according to another embodiment, the disposable cassette comprises an information carrier.

Preferably, according to another embodiment, the information tag is one of an RFID chip, NFC chip, barcode, and QR code.

Preferably, according to another embodiment, the information carrier is attached to an outer surface of the body.

Preferably, according to another embodiment, the information carrier is embedded in the body.

Preferably, according to another embodiment, the information carrier is attached to the medicament container.

Preferably, according to another embodiment, the information carrier is a flexible sheet.

Preferably, according to another embodiment, the disposable cassette comprises a body having a counter fixture releasably attached to the fixture of the housing of the reusable drive unit and a delivery member cover.

Preferably, according to another embodiment, the body extends along an axis between a proximal end and a distal end.

Preferably, according to another embodiment, the delivery member cover is telescopically relative to the proximal end of the body between an extended position where the delivery member cover protrudes from the proximal end of the body in the direction of the axis and a retracted position.

Preferably, according to another embodiment, the delivery member cover comprises a distally directed surface configured to be operably connected to the first switch of the reusable drive unit such that when the delivery member cover is in the retracted position, the first switch is activated.

Preferably, according to another embodiment, the distally directed surface of the delivery member cover is engaged with the proximally directed surface of the activator when the delivery member cover moves from the extended position to the retracted position.

Preferably, according to another embodiment, the distal end of the body of the disposable cassette comprises a distally directed surface configured to be in contact with the activator and thereby moves the activator from the further proximal position to the proximal position when the counter fixture of the body of the disposable cassette is engaged with the fixture of the housing of the reusable drive unit.

Preferably, according to another embodiment, the distally directed surface of the delivery member cover is configured to move the activator from the proximal position to the distal position when the delivery member cover moves from the extended position to the retracted position.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary drugs that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary drugs that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary drugs that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VelP, OFF, GTX, CAV, AD, MAID, AIM, VAC-IE, ADOC, or PE.

Exemplary drugs that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a medicament delivery device with a reusable drive unit of the invention;

FIG. 2 schematically shows a perspective view of components of the reusable drive unit of FIG. 1;

FIG. 3 schematically shows a perspective view of a lead screw of the reusable drive unit of FIG. 1 and a distal nut of the reusable drive unit of FIG. 1;

FIG. 4 schematically shows a cross-section view of the distal nut of FIG. 3 and an inner wall of a housing of the reusable drive unit of FIG. 1;

FIG. 5 schematically shows a perspective view of a driver of the reusable drive unit of FIG. 1.

FIGS. 6-7 schematically show perspective views of a portion of the reusable drive unit of FIG. 1;

FIG. 8 schematically shows a cross-section view of the reusable drive unit of FIG. 1;

FIG. 9 schematically shows a perspective view of a disposable cassette of the medicament delivery device with the reusable drive unit of FIG. 1;

FIG. 10 schematically shows a perspective view of the sub-assembly of FIG. 1 comprises a cap.

### DETAILED DESCRIPTION

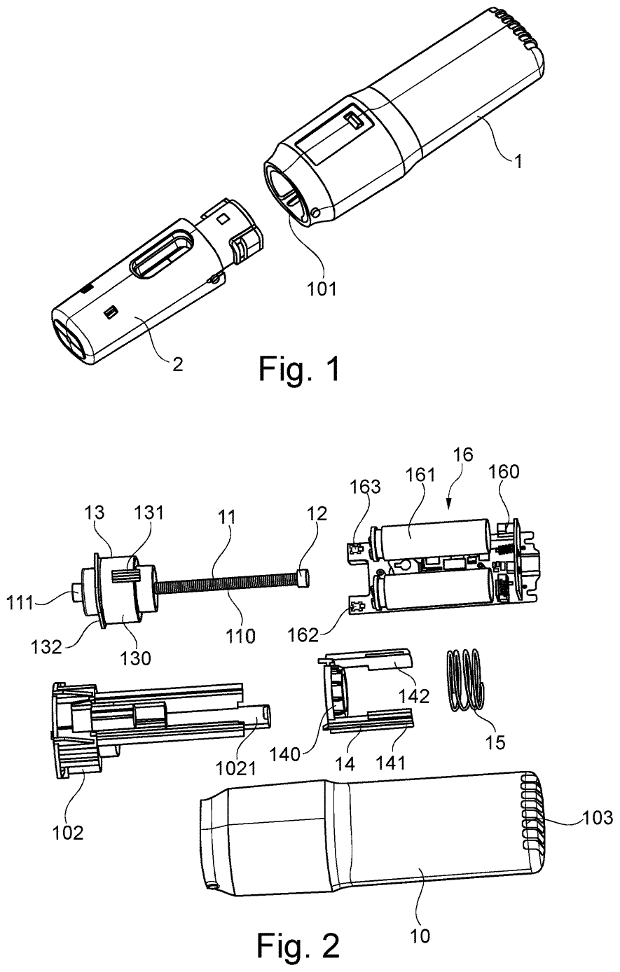

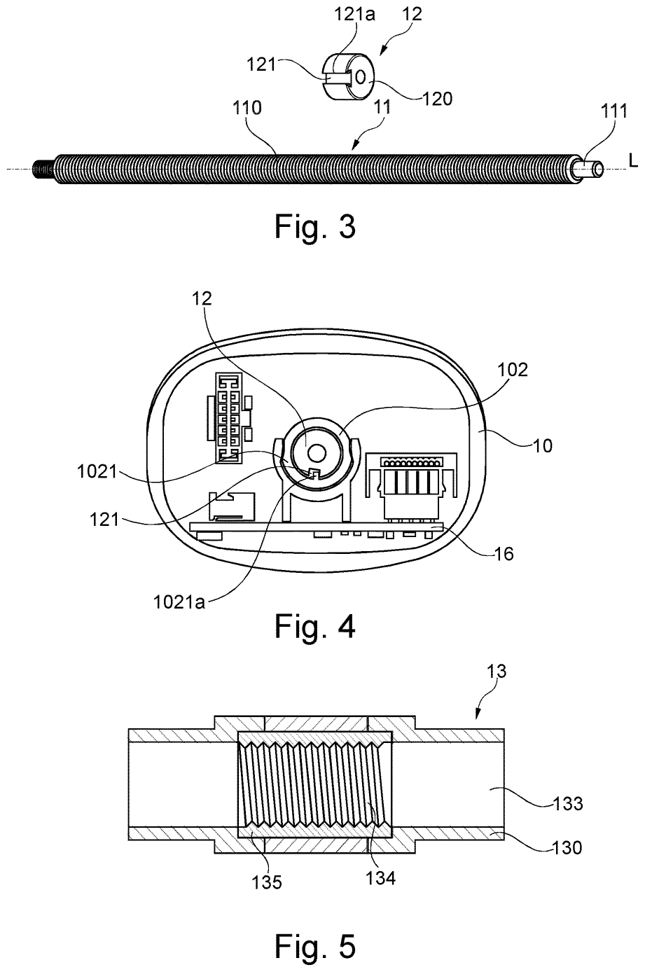

FIGS. 1-10 illustrate a reusable drive unit 1 of a medicament delivery device. The medicament delivery device comprises the reusable drive unit 1 and a disposable cassette 2 releasably attached to the reusable drive unit 1, as shown in FIG. 1. The reusable drive unit 1 comprises a housing 10, a lead screw 11, a distal nut 12, a driver 13 and a first switch 163.

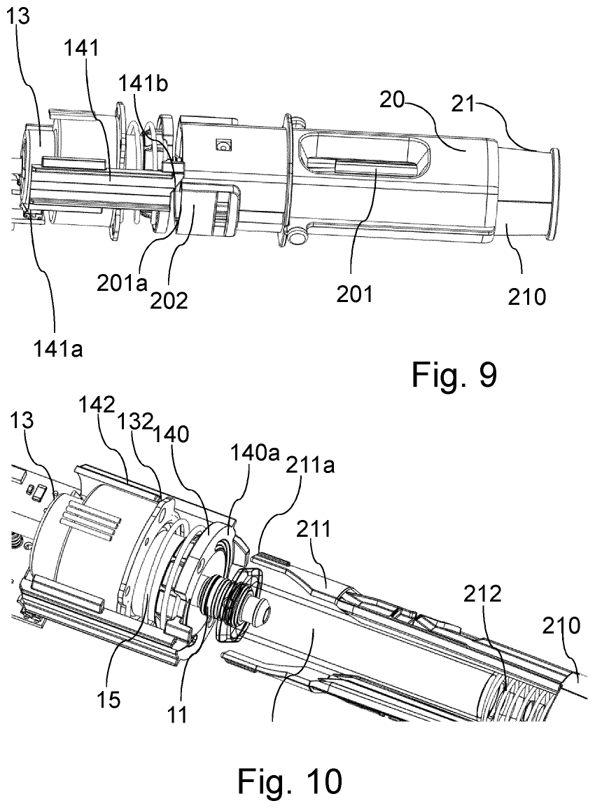

The housing 10 is configured to accommodate a portion of the disposable cassette 2. In one example, the housing 10 comprises a fixture 101 configured to be releasably engaged with the counter fixture 202 of the disposable cassette 2. Preferably, the fixture 101 and the counter fixture 202 form a bayonet engagement, as shown in FIG. 1 and FIG. 9. In this example, the disposable cassette 2 is partially received within the housing 10. Alternatively, the disposable cassette can be fully received within the housing of the reusable drive unit. In this example, the fixture and the counter fixture are not necessary, as the disposable cassette can be made with a shape that is matched with a receiving chamber of the housing of the reusable drive unit.

The housing 10 comprises an electronic section 16. The electronic section 16 comprises a PCB 160 comprising a processor, e.g., MCU or CPU, a battery 161, the first switch 163 and optionally at least one of a second switch 164, a third switch 162, a communication unit, a vibration motor, a buzzer, a camera, a microphone, and a sensor set. The communication unit can be based on wire or wireless telecommunication technology, e.g., RFID, NFC, Bluetooth, Zigbee, LTE, 3G, 4G, 5G, etc. The communication unit can be configured to receive data/information, e.g., an RFID reader/writer, an NFC reader/writer, a Bluetooth receiver, a bar code reader, or an OCR reader, and/or be configured to send out a signal, e.g., Bluetooth Beacon, RFID/NFC transmitter. The sensor set comprises at least one of a gyro sensor, an accelerometer, a temperature sensor and a photo sensor.

In a preferred example, the communication unit comprises an RFID reader/writer and/or an NFC reader/writer. For example, the disposable cassette is arranged with an information tag, e.g., an RFID or NFC tags. In a preferred example, the information tag is pre-programmed with information about the medicament. For example, some treatments require the patient takes multiple different medicaments in a specific order. In this example, the patient receives multiple disposable cassettes containing different medicaments respective; the information tag on each cassette contains the information about the sequence. Thus, when the user, e.g., the patient or a caregiver, attaches the cassette to the reusable drive unit, the communication unit can read the information and if the user does not attach the cassette in the correct order, e.g., the cassette for the second shot is attached rather than the cassette for the first shot is attached, the processor can generate a feedback to the user and/or block at least one of the driver 13, the lead screw 11 and the distal nut 12. In a preferred example, the processor is configured to generate feedback to the user and block the driver 13 if a wrong cassette, e.g., either the cassette is unauthorized or the cassette is in the wrong order, is attached. Thus, the user cannot start the medicament delivery operation. Furthermore, in a preferred example, some treatments require one medicament to be delivered after certain period of time that the first medicament is received. For example, the patient might be prescribed to receive Enhanze® before receiving Hyqvia; and the patient needs to wait 10 minutes for Enhanze® being properly absorbed. In this example, if the user attaches the cassette with Hyqvia first, the user will get warming feedback, e.g., flashing light or sound indication. Furthermore, if the user tries to start the medicament delivery operation, the processor will control and block the movement of the driver 13. In one example where the driver 13 is a motor, the processor is configured to block the signal that is configured to trigger the rotation of the motor as the information from the information tag on the Hyqvia cassette is read by the communication unit. Once the user attaches the cassette with Enhanze®, the processor is configured to enable the trigger signal to reach and start the movement of the motor. As a result, the patient can receive the treatment of Enhanze®. Furthermore, after the medicament delivery of the cassette of Enhanze®, the user will then attach the cassette with Hyqvia to the reusable drive unit. In this example, the processor is configured to control and block the movement of the driver 13 for at least 10 minutes, so that the previous delivered medicament, Enhanze®, can be properly absorbed by the patient.

The housing 10 comprises an inner housing 102. The inner housing 102 comprises an inner wall 1021.

The lead screw 11 extends along a longitudinal axis L between a proximal end and a distal end within the housing 10. In one example, the housing 10 extends along the longitudinal axis L between a proximal end and a distal end, as shown in FIGS. 1-2. Alternatively, the longitudinal axis of the lead screw is transverse to the axis that the housing extends along. The lead screw 11 comprises a thread 110 extending between the proximal end and the distal end. The lead screw 11 comprises a lead screw head 111 configured to be moved into the disposable cassette 2 to expel the medicament contained within the disposable cassette 2. In one example, the inner housing 102 comprises a channel portion configured to guide the movement of the lead screw 11.

The distal nut 12 is configured to rotationally hold the lead screw 11 so that the distal nut 12 and the lead screw 11 cannot rotate around the longitudinal axis L relative to the housing 10.

The distal nut 12 is attached to the distal end of the lead screw 11. The distal nut 12 comprises a surface 121a facing in a direction transverse to the longitudinal axis L. The surface 121a is adjacent to the inner wall 1021 of the housing 10 when the distal nut 12 is moved along the longitudinal axis L with the lead screw 11. The distal nut 12 comprises a non-circular cross-section perpendicular to the longitudinal axis and the inner wall 1021 of the housing 10 comprises a non-circular cross-section perpendicular to the longitudinal axis L such that the distal nut 12 and the lead screw are rotationally fixed to the housing 10. The distal nut comprises an outer surface having the non-circular cross-section perpendicular to the longitudinal axis L; and the inner wall of the housing comprises an inner surface having the non-circular cross-section perpendicular to the longitudinal axis. In one example, the entire outer surface of the distal nut has the non-circular cross-section perpendicular to the longitudinal axis L, namely, the non-circular cross-section can be observed from the distal end of the distal nut and the proximal end of the distal nut. Alternatively, a part of the outer surface of the distal nut has the non-circular cross-section perpendicular to the longitudinal axis L, namely, the non-circular cross-section can only be observed from either the distal end of the distal nut or the proximal end of the distal nut. Similarly, the inner wall of the housing can be arranged that the entire inner surface of the inner wall having the non-circular cross-section perpendicular to the longitudinal axis L; or a part of the entire inner surface of the inner wall having the non-circular cross-section perpendicular to the longitudinal axis L.

As it is common to have a small gap between components during manufacturing for dealing with the tolerance issue, meaning that as the injection molding process might not generate components that are all identical, the components that are designed to be in contact with one another all the time in a product may cause a significant waste for those components that cannot fit into one another; therefore, the term ‘adjacent’ means two objects are ‘nearby’ one another but not necessarily in contact to one another.

In one example, an engagement formed between the distal nut 12 and the inner wall 1021 of the housing 10 is a ribs-ribs engagement or a ribs-grooves engagement. In one example, the distal nut 12 comprises a groove 121 in a nut body 120, and the inner wall 1021 comprises a rib 1021a positioned within the groove 121. The rib 1021a extends in the direction of the longitudinal axis L. When the distal nut 12 is moved along the longitudinal axis L, the groove 121 moves along the rib 1021a. Alternatively, the distal nut comprises a polygonal cross-section perpendicular to the longitudinal axis and the inner wall of the housing comprises a polygonal cross-section perpendicular to the longitudinal axis L When the distal nut is moved along the longitudinal axis L, the distal nut moves along the inner wall. Therefore, the rotation between the housing and the distal nut can be prevented.

In a preferred example, the distal nut can provide a function to determine the dosage to be expelled. In one example, the inner wall comprises a radial protrusion extending towards the longitudinal axis L. The radial protrusion is configured to block the proximal movement of the distal nut, thus to block the proximal movement of the lead screw. Therefore, only a certain amount of the medicament within the disposable cassette of the medicament delivery device can be expelled. In another example, the inner wall comprises multiple radial protrusions that are axially and circumferentially offset relative to one another. In this example, the different deliverable dosages can be determined by using distal nuts of different shapes. For example, a set of distal nuts can be provided. Each of the distal nuts is formed with the cross-section shape as viewed along the longitudinal axis L being different from one another. In this example, each distal nut is configured to be blocked at a different axial position relative to the housing by at least one of the multiple protrusions of the inner wall. For example, one of the distal nuts comprises a triangle cross-section as viewed along the longitudinal axis L; another one of the distal nuts comprises a rectangular cross-section as viewed from the longitudinal axis L. In this example, at least one of the multiple protrusions of the inner wall of the housing is configured to block the rectangular distal nut in a position; but the at least one of the multiple protrusions is configured to be spaced apart from the triangle distal nut. Thus, the triangle distal nut can be blocked in another position that is more proximal than the rectangular distal nut.

It should be noted that, as the reusable drive unit 1 can control the deliverable dosage by controlling the motor, using the distal nut to provide the function of determining the dosage to be expelled is optional. In one example where the distal nut provides the function of determining the dosage, the housing can be universal, and the moving distance of the lead screw can be limited by using different distal nuts. The hard stop of the lead screw can be used as a safety mechanism to limit the maximum derivable dose of the medicament to the patients. Thus, even if the electronic section 16 is programmed with incorrect medicament information, e.g., error programming by the user or the disposable cassette 2 comprises a wrong tag, e.g., by mistake in the production line, the risk of overdose can be reduced.

The driver 13 is rotatable around the lead screw 11 within the housing 10. The driver 13 is engaged with the lead screw 11 via a threaded engagement. The driver 13 is connected to the electronic section 16 such that the electronic section 16 controls the rotation of the driver 13. When the driver 13 rotates, the torque is transferred to the lead screw 11 via the thread engagement between the lead screw 11 and the driver 13. In one example, the drive 13 is connected to the electronic section 16 via a wire 131. As the lead screw 11 is rotationally fixed to the housing 10, the torque from the driver 13 is converted to an axial movement of the lead screw 11. In a preferred example, the driver 13 is a motor electrically connected to the first switch 163. In a preferred example, the driver 13 is a stepper motor. As shown in FIG. 5, the motor comprises a rotor 130 and a channel 133 extending through the rotor 130. The lead screw 11 is partially positioned within the channel 133. In a preferred example, the longitudinal axis L is the central axis of the motor. In one example, the channel comprises a thread 134 extending around the channel. The thread is engaged with the thread of the lead screw. Alternatively, the reusable drive unit comprises a connector 135 operably connected to the rotor 130 such that the connector 135 is configured to be rotated by the rotor. In this example, a channel extends through the connector and is aligned with the channel of the rotor 130 along the longitudinal axis L. The channel of the connector 135 comprises a thread 134 extending around the channel of the connector 135. The thread 134 is engaged with the thread of the lead screw 11. In this example, the connector 135 is attached to the rotor 130. In a preferred example, the connector 135 and the rotor 130 are made of different materials. As the thread 134 of the connector 135 is configured to engage with the lead screw 11, the connector 135 can be selected with a material that will create low friction. In one example, the rotor 130 is made of metal and the lead screw 11 is made of metal, then the connector 135 is preferably to be made of plastic.

It should be noted that as the lead screw 11 is rotationally fixed to the housing 10 by the distal nut 12, the lead screw 11 can be standardized. In other words, the shape of the entire lead screw does not need to be customized to provide the rotational prevention function between the lead screw and the housing.

The first switch 163 arranged within the housing 10. The first switch 163 is configured to be used to turn on the driver 13. Upon activation of the first switch 163, the rotation of the driver 13 around the lead screw 11 is configured to move the lead screw 11 proximally so that a stopper of a medicament container M in the disposable cassette 2 is moved by the lead screw 11. Additionally, the first switch can be used to switch on the entire electronic section 16. Alternatively, the first switch can be used to switch the electronic section 16 from a low power consumption mode to a normal working mode.

The first switch can be manually activated by a user of the medicament delivery device. In one example, the first switch is a button protruding from an outer surface of the housing. Alternatively, the housing comprises a touch panel, and the first switch is an electronic switch that can be activated via the touch panel. Alternatively, the reusable drive unit 1 comprises an activator 14 movable relative to the housing 10. In a preferred example, the activator 14 is movable relative to the housing 10 in the direction of the longitudinal axis between a proximal position where the first switch 163 is deactivated and a distal position where the first switch 163 is activated by the activator 14. In one example, the activator 14 comprises an activator body 140, a first arm 142 and a second arm 141. Both the first arm 142 and the second arm 141 are extending from the activator body 140, as shown in FIG. 2. In one example, the reusable drive unit 1 comprises a biasing member 15 configured to move the activator 14 back to its original position.

In one example, as shown in FIG. 7, the activator 14 comprises a first surface 142a. The first surface 142a of the activator 14 is engaged with the first switch 163 in the distal position and is disengaged with the first switch 163 in the proximal position. In one example, the first arm 142 comprises the first surface 142a.

In a preferred example, the activator 14 is configured to be moved from the proximal position to the distal position by a component of the disposable cassette 2 of the medicament delivery device when the disposable cassette is attached to the housing 10. In one example, the activator 14 is configured to be moved from the proximal position to the distal position by a delivery member cover 21 of the disposable cassette 2 of the medicament delivery device when the disposable cassette is attached to the housing 10. In this example, when the user presses the delivery member cover 21 on a medicament delivery site to reveal a medicament delivery member of the medicament delivery device, the axial movement of the delivery member cover 21 moves the activator 14 from the proximal position to the distal position. Therefore, the first switch 163 is activated. In this example, the biasing member 15 is preferably positioned between the activator body 140 and a flange 132 of the driver 13. In one example, the biasing member 15 is a compression spring extending along the longitudinal axis L. In this example, when the medicament delivery operation is completed, the delivery member cover 21 no longer acts on the activator 14, and the biasing member 15 moves the activator 14 back to its original position such that the first switch 163 can be deactivated. In a preferred example, the delivery member cover 21 is biased in the proximal direction relative to the body 20 by a delivery member cover biasing member 212, e.g., a spring or a flexible arm. In this example, once the delivery member cover 21 is lifted from the medicament delivery site, the delivery member cover 21 is biased to cover the delivery member.

Alternatively, the first switch can be a non-contact switch, for example, a reed switch or a light beam. In this example, the first switch does not need to be moved for being activated. In this example, the activator does not need to engage with the first switch to activate the first switch. Therefore, the first surface is not necessary in this example. For example, the activator comprises a magnet so that when the activator is adjacent to the first switch, the first switch is activated. Alternatively, when the activator is moved across the light beam, the first switch is activated.

In a preferred example, the driver 13 is configured to reciprocate the movement of the lead screw 11. In one example where the driver 13 is a motor, the motor can be programmed that it should rotate in an opposite direction once a raised resistance is detected (for example, by detecting the increased current). Alternatively, the driver 13 is designed that will only deliver a preset dose such that once the preset dose is delivered, the motor will rotate in an opposite direction. Alternatively, the driver 13 is configured to rotate in an opposite direction once the first switch is deactivated.

Therefore, when the first switch 163 is activated, the rotor 130 of the motor starts to rotate and thus causes the lead screw 11 to expel the medicament out of the disposable cassette 2. When the medicament container M is empty, the movement of the lead screw 11 towards an exit of the medicament container M will be blocked by the medicament container M. Thus, an increased resistance can be detected and thus the motor can start to rotate in an opposite direction. For example, if the motor is rotated in a clockwise direction around the longitudinal axis L to cause the medicament to be expelled, when increased resistance is detected, the motor can rotate in a counter-clockwise direction around the longitudinal axis L. Therefore, as the lead screw 11 is rotationally fixed to the housing 10, the lead screw 11 is moved distally.

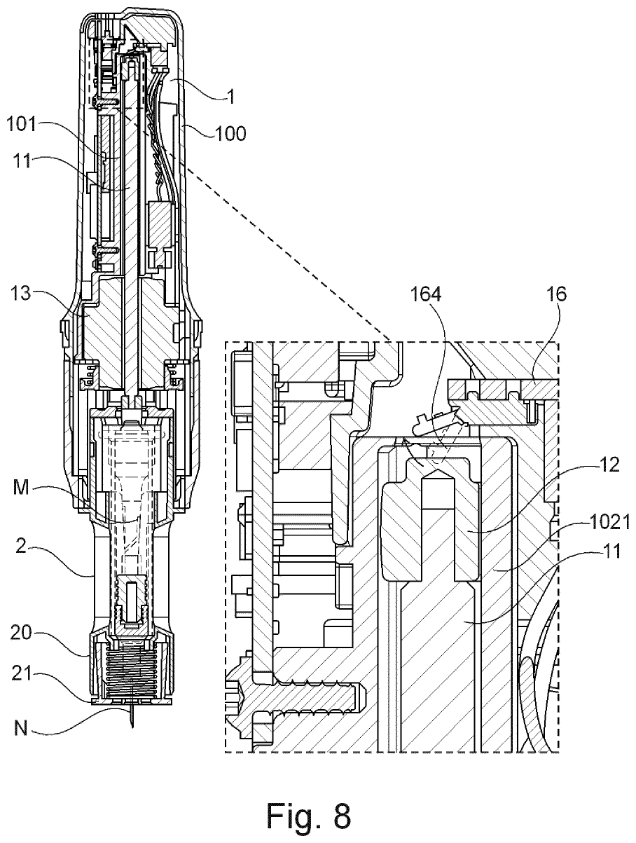

In one example where the reusable drive unit 1 comprises the second switch 164 accommodated within the housing 10, as shown in FIG. 8. The second switch 164 is aligned with the distal nut 12 in the direction of the longitudinal axis L. The second switch 164 is positioned distally away from the distal nut 12. The second switch 164 is configured to be switched by the distal nut 12 when the lead screw is moved distally relative to the driver such that when the second switch is switched the motor is stopped. Therefore, at the end of the medicament delivery operation, the lead screw 11 is moved in the distal direction relative to the housing 10 by the motor as mentioned above, the motor keeps rotating until the second switch 164 is switched off by the distal nut 12. In a preferred example, when the second switch 164 is switched by the distal nut 12, the second switch 164 is configured to switch off the entire electronic section 16. Alternatively, the second switch 164 is configured to turn the electronic section 16 into the low power consumption mode.

In one example, the second switch is configured to be switched by being pushed in the distal direction relative to the housing by the distal nut. Additionally, the second switch comprises a flipping arm. The second switch 164 is configured to be switched when the flipping arm is pivoted by the distal nut 12, as shown in FIG. 8. In this example, the second switch 164 optionally comprises a flexible member, e.g., a spring or a flexible arm, configured to push the second switch 164 towards the distal nut 12 such that when the distal nut 12 is moved apart from the second switch 164, the second switch 164 is moved to its original position. Alternatively, the second switch is a reed switch, and the distal nut comprises a magnet. Therefore, the distal nut does not need to be physically in contact with the second switch to switch the second switch.

Furthermore, in one example, the reusable drive unit 1 comprises a third switch. In one example, the third switch is configured to switch on the entire electronic section 16. Alternatively, the first switch can be used to switch the electronic section 16 from a low power consumption mode to a normal working mode. Additionally, in one example where the reusable drive unit 1 comprises the communication unit, the communication unit is connected to the third switch. In this example, the communication unit is configured to be activated when the third switch is activated. In a preferred example, the third switch is configured to be operably activated by a component of the disposable cassette of the medicament delivery device when the disposable cassette is attached to the housing. Therefore, the medicament delivery device can be automatically switched on or moved to a working mode by attaching the disposable cassette 2 to the housing 10.

In one example, the third switch 162 is operably movable by a component of the disposable cassette of the medicament delivery device when the disposable cassette is attached to the housing between an active position where the third switch is activated and an inactive position where the third switch is deactivated. Alternatively, the third switch is a non-contact switch, e.g., a reed switch, a light beam and/or an induction arrangement. In one example where the third switch is a non-contact switch, the third switch does not need to be moved to be activated and also does not need to be contacted by the component of the disposable cassette. For example, the body 20 of the disposable cassette 2 comprises a magnet so that when the body 20 of the disposable cassette 2 is at least received within the housing 10 of the reusable drive unit 1, the third switch 162 is activated.

In one example where reusable drive unit 1 comprises the activator 14. The activator 14 optionally comprises a second surface 141a. The activator 14 is movable in the direction of the longitudinal axis between a further proximal position where the second surface 141a of the activator 14 is spaced apart from the third switch and the proximal position where the second surface 141a of the activator 14 is engaged with the third switch 162 such that the third switch 162 is activated. In one example, the second arm 141 of the activator 14 comprises the second surface 141a.

In one example, the activator 14 comprises a proximally directed surface 141b configured to be in contact with a distally directed surface 201a of the component of the disposable cassette 2. In a preferred example, the proximally directed surface 141b of the activator 14 is bevelled, as shown in FIG. 9. In one example, the second arm 141 of the activator 14 comprises the proximally directed surface 141b. Similarly, when the user removes the disposable cassette 2 from the housing 10, the biasing member 15 can move the activator 14 back to its original position. Therefore, the third switch 162 can be deactivated. In one example, the first surface 142a is positioned proximally relative to the second surface 141a, as shown in FIG. 7.

Another aspect of the invention that provides a medicament delivery device comprises the reusable drive unit 1 and the disposable cassette 2. The disposable cassette 2 comprises a body 20 having the counter fixture 202 releasably attached to the fixture 101 of the housing 10 of the reusable drive unit 1 and the delivery member cover 21.

The body 20 extends along an axis between a proximal end and a distal end. The delivery member cover 21 comprises a proximal portion 210 and a distal portion 211.

The delivery member cover 21 is telescopically relative to the proximal end of the body 20 between an extended position where the delivery member cover 21 protrudes from the proximal end of the body 20 in the direction of the axis, as shown in FIG. 9, and a retracted position. The delivery member cover 21 comprises a distally directed surface 211a configured to operably connected to the first switch 163 of the reusable drive unit 1 such that when the delivery member cover 21 is in the retracted position, the first switch 163 is activated. The proximal portion 210 is configured to cover the medicament delivery member of the medicament delivery device. In a preferred example, the proximal portion 210 is tubular. In one example, the proximal portion 210 comprises a proximal flange configured to be in contact with the medicament delivery site. Therefore, when the user presses the delivery member cover 21 against the medicament delivery site, the pressure can be evenly distributed on the medicament delivery site. In a preferred example, the flange is configured to contact a proximal end edge of the housing 10 when the delivery member cover 21 is in the retracted position. In a preferred example, the distal portion 211 comprises the distally directed surface 211a.

In one example, the distally directed surface 211a of the delivery member cover 21 is engaged with the proximally directed surface 140a of the activator 14 when the delivery member cover 21 moves from the extended position to the retracted position.

In one example, the distal end of the body 20 of the disposable cassette 2 comprises a distally directed surface 201a configured to in contact with the activator 14 and thereby moves the activator 14 from the further proximal position to the proximal position when the counter fixture 202 of the body 20 of the disposable cassette 2 is engaged with the fixture 101 of the housing 10 of the reusable drive unit 1. Optionally, the body of the disposable cassette 2 comprises a window 201. In this example, the user can observe the contained medicament through the window 201.

In one example where the proximally directed surface 141b of the activator 14 is bevelled and the fixture 101 and the counter fixture 202 forms a bayonet connection, the rotation of the disposable cassette 2, relative to the housing 10 namely, the movement that causes the disposable cassette 2 to be attached to the housing 10, causes the activator 14 to move axially to the proximal position of the activator 14.

Furthermore, in one example, the distally directed surface 211a of the delivery member cover 21 is configured to move the activator 14 from the proximal position to the distal position when the delivery member cover 21 moves from the extended position to the retracted position.

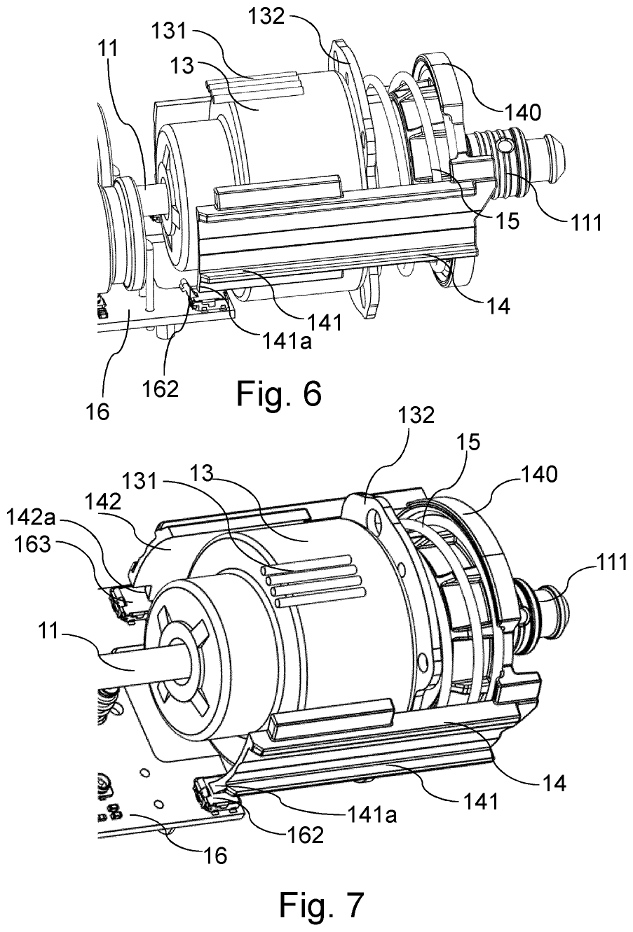

In one example, the medicament delivery device is operated with the following steps. In this example, the disposable cassette 2 comprises an information carrier.
1. The user attaches the disposable cassette 2 to the housing 10 of the reusable drive unit 1, such that the disposable cassette 2 moves the activator 14, and therefore, the third switch 162 is activated by such disposable cassette 2 insertions, as shown in FIG. 6.
2. The activation of the third switch 162 causes the electronic section 16 to be switched on, including the communication unit.
3. The communication unit receives the information carried by the disposable cassette 2, e.g., the information related to the medicament contained within the disposable cassette.
4. The electronic section 16 provides at least one of the operations: sending the information to the cloud database and/or personal smart device, providing an indication to the user, e.g., via the indicator 103, verifying the information from the disposable cassette 2, determining the medicament delivery profile, such as the medicament delivery speed and/or dosage, recording the use of the disposable cassette 2.
5. The user presses the delivery member cover 21 against the medicament delivery site.
6. The movement of the delivery member cover 21 moves the activator 14 further such that the first surface 142a activates the first switch 163.
7. The driver 13 starts to rotate as the first switch 163 is activated. Therefore, the lead screw 11 moves proximally into the disposable cassette 2 to expel the medicament.
8. When a pre-set dosage (by programmed or determined based on the information from the disposable cassette 2) is delivered or the medicament container M is empty, the driver 13 moves the lead screw 11 in the distal direction relative to the housing 10.
9. The second switch 164 is switched by the distal nut 12. Therefore, the driver 13 stops.
10. The user removes the disposable cassette 2 from the housing 10. As the disposable cassette 2 no longer supports the activator 14 to keep the third switch 162 in the activated position, the biasing member 15 moves the activator 14 to its original position such the third switch 162 is moved to its inactive position. Therefore, the electronic section 16 is switched off.

Furthermore, in one example where the communication unit comprises an RFID reader/writer and/or an NFC reader/writer, the post-administration information can be written to the RFID/NFC tag on the disposable cassette. For example, whether the cassette is now empty or there is residual dose.

Furthermore, the housing of the medicament delivery device, as mentioned in any example, may be provided with (i.e., molded in, molded with) a compound featuring persistently antimicrobial, antifungal, and/or antiviral properties. Alternatively, a compound featuring persistently antimicrobial, antifungal, and/or antiviral properties may be applied to the molded (i.e., finished) components through secondary processes (e.g., chemical vapor deposition), spraying, or dipping processes.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are defined by the following clauses.
1. A reusable drive unit (1) of a medicament delivery device, wherein the medicament delivery device comprises the reusable drive unit (1) and a disposable cassette (2) releasably attached to the reusable drive unit (1), the reusable drive unit (1) comprising:
   a housing (10) configured to accommodate a portion of the disposable cassette (2);
   wherein the housing (10) comprises an inner wall (1021);
   a lead screw (11) extending along a longitudinal axis (L) between a proximal end and a distal end within the housing (10); wherein the lead screw (11) comprises a thread (110) extending between the proximal end and the distal end;
   a distal nut (12) attached to the distal end of the lead screw (11); wherein the distal nut (12) comprises a surface (121a) facing in a direction transverse to the longitudinal axis (L); wherein the surface (121a) is adjacent to the inner wall (1021) of the housing (10) when the distal nut (12 moves along the longitudinal axis (L); wherein the distal nut (12) comprises an outer surface having a non-circular cross-section perpendicular to the longitudinal axis (L) and the inner wall (1021) of the housing (10) comprises an inner wall having a non-circular cross-section perpendicular to the longitudinal axis (L) such that the distal nut (12) and the lead screw are rotationally fixed to the housing (10);
   a driver (13) rotatable around the lead screw (11) within the housing (10); wherein the driver (13) is engaged with the lead screw (11) via a thread engagement;
   a first switch (163) arranged within the housing (10);
   wherein upon activation of the first switch (163), the rotation of the driver (13) around the lead screw (11) is configured to move the lead screw (11) proximally so that a stopper of a medicament container (M) in the disposable cassette (2) is moved by the lead screw (11).
2. The reusable drive unit according to clause 1, wherein the driver is a motor electrically connected to the first switch.
3. The reusable drive unit according to clause 2, wherein the motor comprises a rotor and a channel extending through the rotor; and wherein the lead screw is partially positioned within the channel.
4. The reusable drive unit according to clause 3, wherein the channel comprises a thread extending around the channel; and wherein the thread is engaged with the thread of the lead screw.
5. The reusable drive unit according to clause 3, comprising a connector operably connected to the rotor such that the connector is configured to be rotated by the rotor; wherein a channel extends through the connector and is aligned with the channel of the rotor along the longitudinal axis; wherein the channel of the connector comprises a thread extending around the channel of the connector; and wherein the thread is engaged with the thread of the lead screw.
6. The reusable drive unit according to clause 5, wherein the connector is attached to the rotor.
7. The reusable drive unit according to clause 5 or 6, wherein the connector and the rotor are made of different material.
8. The reusable drive unit according to clause 7, wherein the connector is made of plastic; and wherein the rotor is made of metal.
9. The reusable drive unit according to any of clauses 2-8, wherein the longitudinal axis is the central axis of the motor.
10. The reusable drive unit according to any of the preceding clauses, wherein an engagement formed between the distal nut and the inner wall of the housing is a ribs-ribs engagement or a ribs-grooves engagement.
11. The reusable drive unit according to any of the preceding clauses, wherein the distal nut comprises a polygonal cross-section perpendicular to the longitudinal axis and the inner wall of the housing comprises a polygonal cross-section perpendicular to the longitudinal axis.
12. The reusable drive unit according to any of the preceding clauses, wherein the driver is configured to reciprocate the movement of the lead screw.
13. The reusable drive unit according to clause 12, comprising a second switch accommodated within the housing; wherein the second switch is aligned with the distal nut of the lead screw in the direction of the longitudinal axis; wherein the second switch is positioned distally away from the distal nut of the lead screw; and wherein the second switch is configured to be switched by the distal nut when the lead screw is moved distally relative to the driver such that when the second switch is switched the motor is stopped.
14. The reusable drive unit according to clause 13, wherein the second switch is configured to be switched by being pushed in the distal direction relative to the housing by the distal nut.
15. The reusable drive unit according to clause 13, wherein the second switch comprises a flipping arm; and wherein the second switch is configured to be switched when the flipping arm is pivoted by the distal nut.
16. The reusable drive unit according to any of the preceding clauses, wherein the housing extends along the longitudinal axis between a proximal end and a distal end; and wherein the housing comprises a fixture at a proximal portion of the housing; wherein the fixture is configured to be releasably attached to a counter fixture of the disposable cassette of the medicament delivery device.
17. The reusable drive unit according to any of the preceding clauses, comprising an activator movable relative to the housing in the direction of the longitudinal axis between a proximal position where the first switch is deactivated and a distal position where the first switch is activated by the activator.
18. The reusable drive unit according to clause 17, wherein the activator comprises a first surface; wherein the first surface of the activator is engaged with the first switch in the distal position and is disengaged with the first switch in the proximal position.
19. The reusable drive unit according to clause 17 or 18, wherein the activator is configured to be moved from the proximal position to the distal position by a component of the disposable cassette of the medicament delivery device when the disposable cassette is attached to the housing.
20. The reusable drive unit according to any of the preceding clauses, comprising a third switch and a communication unit connected to the third switch; wherein the communication unit is configured to be activated when the third switch is activated; and wherein the third switch is configured to be operably activated by a component of the disposable cassette of the medicament delivery device when the disposable cassette is attached to the housing.
21. The reusable drive unit according to clause 20, the third switch is operably movable by a component of the disposable cassette of the medicament delivery device when the disposable cassette is attached to the housing between an active position where the third switch is activated and an inactive position where the third switch is deactivated.
22. The reusable drive unit according to clause 21 when dependent on any of clauses 17-19, wherein the activator comprises a second surface; wherein the activator is movable in the direction of the longitudinal axis between a further proximal position where the second surface of the activator is spaced apart from the third switch and the proximal position where the second surface of the activator is engaged with the third switch such that the third switch is activated.
23. A medicament delivery device comprises the reusable drive unit according to any of the preceding clauses, wherein the medicament delivery device comprises a disposable cassette comprising a body having a counter fixture releasably attached to the fixture of the housing of the reusable drive unit and a delivery member cover;
   wherein the body extends along an axis between a proximal end and a distal end;
   wherein the delivery member cover is telescopically relative to the proximal end of the body between an extended position where the delivery member cover protrudes from the proximal end of the body in the direction of the axis and a retracted position; and wherein the delivery member cover comprises a distally directed surface configured to operably connected to the first switch of the reusable drive unit such that when the delivery member cover is in the retracted position, the first switch is activated.
24. The medicament delivery device according to clause 24 when dependent on any of clauses 18-19, wherein the distally directed surface of the delivery member cover is engaged with the proximally directed surface of the activator when the delivery member cover moves from the extended position to the retracted position.
25. The medicament delivery device according to clause 23 or 25 when dependent on a combination of clause 22 and any of clauses 19-20, wherein the distal end of the body of the disposable cassette comprises a distally directed surface configured to in contact with the activator and thereby moves the activator from the further proximal position to the proximal position when the counter fixture of the body of the disposable cassette is engaged with the fixture of the housing of the reusable drive unit.
26. The medicament delivery device according to a combination of clause 24 and 25, wherein the distally directed surface of the delivery member cover is configured to move the activator from the proximal position to the distal position when the delivery member cover moves from the extended position to the retracted position.

1-15. (canceled)

16. A reusable drive unit of a medicament delivery device, wherein the medicament delivery device comprises the reusable drive unit and a disposable cassette releasably attached to the reusable drive unit, the reusable drive unit comprising:
a housing configured to accommodate a portion of the disposable cassette, wherein the housing comprises an inner wall;
a lead screw extending along a longitudinal axis between a proximal end and a distal end within the housing, wherein the lead screw comprises a thread extending between the proximal end and the distal end;
a distal nut attached to the distal end of the lead screw, wherein the distal nut comprises a surface facing in a direction transverse to the longitudinal axis, wherein the surface is adjacent to the inner wall of the housing when the distal nut moves along the longitudinal axis, wherein the distal nut comprises an outer surface having a non-circular cross-section perpendicular to the longitudinal axis and the inner wall of the housing comprises an inner wall having a non-circular cross-section perpendicular to the longitudinal axis such that the distal nut and the lead screw are rotationally fixed to the housing;
a driver rotatable around the lead screw within the housing, wherein the driver is engaged with the lead screw via a thread engagement; and
a first switch arranged within the housing,
wherein upon activation of the first switch, the rotation of the driver around the lead screw is configured to move the lead screw proximally so that a stopper of a medicament container in the disposable cassette is moved by the lead screw.

17. The reusable drive unit according to claim 6, wherein the driver is a motor electrically connected to the first switch.

18. The reusable drive unit according to claim 17, wherein the motor comprises a rotor and a channel extending through the rotor, and wherein the lead screw is partially positioned within the channel.

19. The reusable drive unit according to claim 18, further comprising:
a connector operably connected to the rotor such that the connector is configured to be rotated by the rotor, wherein a channel extends through the connector and is aligned with the channel of the rotor along the longitudinal axis, wherein the channel of the connector comprises a thread extending around the channel of the connector, and wherein the thread is engaged with the thread of the lead screw.

20. The reusable drive unit according to claim 19, wherein the connector and the rotor are made of different material.

21. The reusable drive unit according to claim 16, wherein an engagement formed between the distal nut and the inner wall of the housing is a ribs-ribs engagement or a ribs-grooves engagement.

22. The reusable drive unit according to claim 16, wherein the distal nut comprises a polygonal cross-section perpendicular to the longitudinal axis and the inner wall of the housing comprises a polygonal cross-section perpendicular to the longitudinal axis.

23. The reusable drive unit according claim 16, wherein the driver is configured to reciprocate the movement of the lead screw.

24. The reusable drive unit according to claim 23, further comprising:
a second switch accommodated within the housing, wherein the second switch is aligned with the distal nut of the lead screw in the direction of the longitudinal axis, wherein the second switch is positioned distally away from the distal nut of the lead screw, and wherein the second switch is configured to be switched by the distal nut when the lead screw is moved distally relative to the driver such that when the second switch is switched the motor is stopped.

25. The reusable drive unit according to claim 16, wherein the housing extends along the longitudinal axis between a proximal end and a distal end, and wherein the housing comprises a fixture at a proximal portion of the housing, wherein the fixture is configured to be releasably attached to a counter fixture of the disposable cassette of the medicament delivery device.

26. The reusable drive unit according to claim 16, comprising an activator movable relative to the housing in the direction of the longitudinal axis between a proximal position where the first switch is deactivated and a distal position where the first switch is activated by the activator.

27. The reusable drive unit according to claim 26, wherein the activator comprises a first surface, and wherein the first surface of the activator is engaged with the first switch in the distal position and is disengaged with the first switch in the proximal position.

28. The reusable drive unit according to claim 26, wherein the activator is configured to be moved from the proximal position to the distal position by a component of the disposable cassette of the medicament delivery device when the disposable cassette is attached to the housing.

29. The reusable drive unit according to claim 16, further comprising:
a third switch and a communication unit connected to the third switch, wherein the communication unit is configured to be activated when the third switch is activated, and wherein the third switch is configured to be operably activated by a component of the disposable cassette of the medicament delivery device when the disposable cassette is attached to the housing.

30. The reusable drive unit according to claim 29, wherein the third switch is operably movable by a component of the disposable cassette of the medicament delivery device when the disposable cassette is attached to the housing between an active position where the third switch is activated and an inactive position where the third switch is deactivated.
